(19) United States
(12) Patent Application Publication
(10) Pub. No.: US 2014/0324035 A1
(43) Pub. Date: Oct. 30, 2014

(54) APPARATUS FOR TREATING INCISIONS

(71) Applicant: LUMENIS LTD., Yokneam Ilit (IL)

(72) Inventors: Yoni Iger, Yoqneam (IL); Dan Albeck, Givat Shmuel (IL)

(21) Appl. No.: 14/366,072

(22) PCT Filed: Dec. 18, 2012
(86) PCT No.: PCT/IB2012/057416

§ 371 Date: Jun. 17, 2014

Related U.S. Application Data
(60) Provisional application No. 61/577,754, filed on Dec. 20, 2011.

(51) Int. Cl. A61B 18/08; A61B 18/14; A61N 7/00; A61B 18/20

(52) U.S. Cl. CPC A61B 18/082; A61B 18/203; A61B 18/1477; A61N 7/00; A61B 2018/0063
USPC 606/13; 606/28; 606/49

(57) ABSTRACT

A system and device for treating a target tissue is provided herein. The treating device and system is based on a fractional treatment of an incision edge prior to closure of the incision to prevent scar formation.

### TECHNICAL FIELD

The present invention relates to fractional tissue treatment and more particularly, to fractional treatment of an incision to prevent scar formation. Skin integrity loss triggers healing and regeneration processes of the tissue. Awry healing process may result in an exuberance of fibroblastic proliferation leading to wound-confined hypertrophic scar while further exuberance can result in keloid scar formation which extends beyond wound boundaries. These scars may be itchy or painful in some individuals and may cause aesthetic problem. Surgical incisions and other wounds are sources for skin integrity loss which may trigger such a healing and regeneration processes and which may lead to scar formation.

### SUMMARY OF THE INVENTION

The present invention provides a system for treating incisions, for the prevention of scar tissue, the system comprising: a wedge body, the wedge body having an upper surface, a lower surface and a volume, wherein the lower surface is configured to contact at least a portion of an incision edge; and wherein the wedge body volume is configured to deliver treatment energy through the lower wedge surface to adjacent incision edges.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout.

In the accompanying drawings:

FIG. 1 is a high level schematic illustration of a wedge body,

FIG. 2 is a high level schematic illustration of one portion of a lower surface of the wedge body.

FIG. 3 illustrates another aspect of the present invention in which, at least in one embodiment, a hot body is disclosed.

FIG. 4 illustrates another embodiment wherein the hot tip body comprises at least one rotating element.

FIG. 5 illustrates a further embodiment wherein the hot tip body comprises other rotating means.

FIG. 6 illustrates another embodiment of the present invention comprising a shaft.

The drawings together with the following detailed description make apparent to those skilled in the art how the invention may be embodied in practice.

### DETAILED DESCRIPTION

Prior to setting forth the detailed description, it may be helpful to set forth definitions of certain terms that will be used hereinafter.

The term “electrode” as used herein in this application refers to any type of energy transmitting element or energy irradiating element or energy delivering element. These elements for example, can be among other things radiofrequency electrode, light emitting diodes, laser diodes, optical fibers, ultrasound transducers, micro-needle electrodes, hot tips etc.

The term “fractional treatment” as used herein in this application refers to a treatment of a target tissue or organ in which at least one treatment point is created in the tissue and is surrounded by a non-treated tissue. On a target tissue, one or more treatment points may be created in a variety of sizes, depths, patterns and densities. Fractional treatment may be invasive, non-invasive or any combination of the two.

The term “energy source” as used herein in this application refers to any energy source which may create fractional treatment. As non limiting examples for such energy sources are laser, non-coherent light sources, radio frequency generators, microwave generators, cryogenically cooled material, ultrasound, heat generators etc.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

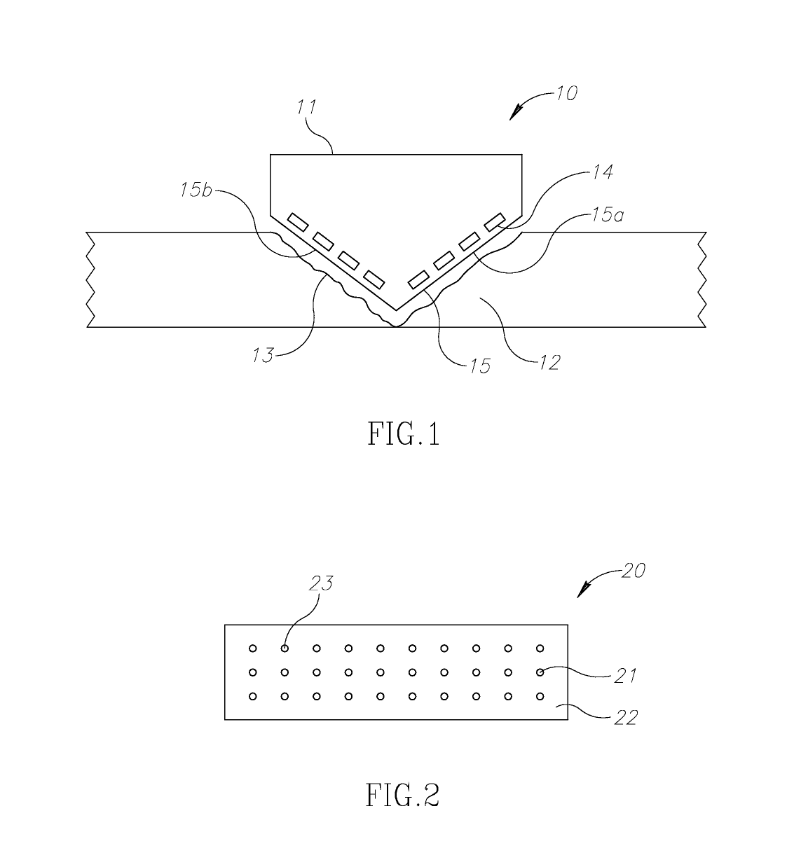

FIG. 1 illustrates a wedge body 10 according to one embodiment of the present invention. The wedge body 10 consists of a volumetric element having an upper surface 11 and a lower surface 15. The lower surface 15 of the wedge body 10 is configured to contact at least a portion of the incision edges 12 and 13. In another embodiment of the invention, the lower surface 15 may further define a first region 15a, configured to contact at least a portion of a first incision edge 12 and a second region 15b, configured to contact at least a portion of a second incision edge 13. The lower surface 15 may be designed by a skilled man in the art in many different ways and shapes in order to effectively fill the gap between the incision edges 12 and 13 and create an effective contact with incision edges 12 and 13 to allow an effective energy delivery from wedge body 10 to at least portion of the incision edges 12 and 13.

According to one embodiment of the present invention, the wedge body lower surfaces 15a and 15b may define a triangular shape. In yet another embodiment of the present invention, this wedge body lower surface may comprise of more than two surfaces to define any polygon shape or it can even be round.

The material of the wedge body can be rigid or flexible and should be biocompatible and of a medical graded material. According to one embodiment of the present invention, the wedge body 10 is rigid and made of a medical grade plastic. In yet another embodiment of the present invention, the wedge body 10 may be flexible so it can more precisely conform its lower surface 15 to be accommodated between incision edges 12 and 13 and to more effectively establish a contact with incision edges 12 and 13. This allows an effective delivery of the treatment energy to achieve the required fractional clinical effect. In order to increase the clinical effect, according to another embodiment of the present invention, the wedge body 10 further comprises a cooling element which allows cooling of the non target tissue.

As illustrated in FIG. 2, according to one embodiment of the invention, the lower surface 15 may comprise at least one treatment zone 20 which is configured to establish a contact between portions of the target tissue and energy sources 21. In yet another configuration, the area 22 between the energy ports 21 may be used to cool the tissue during treatment to allow delivery of higher energies through energy ports 21 to increate tissue efficacy or reduce treatment time.

FIG. 1 illustrates multiple energy sources 14 which can be used according to different embodiments of the invention. According to one embodiment, a diode laser or a strip of diode lasers may be placed within the wedge body volume. In yet another embodiment, arrays of diode lasers may be placed within the wedge body volume. The lower surface 15 is configured to establish optical coupling with at least a portion of the target tissue. In some embodiments of the present invention, the lower surface 15 may have additional optical elements such as lenses to further focus optical energy emitted from the diode lasers onto the target tissue. In yet other embodiments of the present invention, other energy sources may be placed within the wedge body volume 10. These energy sources may be, for example, radiofrequency electrodes, microwave electrodes, ultrasound transducers. In yet another embodiment of the invention, the energy sources may be located in a separated main console external to the wedge body volume. In such an embodiment, the energy source is coupled to the wedge system with a cable.

Another aspect of the present invention may include a disposable shielding element to shield wedge body 10 to allow safer usage of the invention and to reduce costs and complications associated with contamination. In yet another embodiment of the present invention the entire wedge body can be a single patient element with safety features to allow monitoring the number of usage by using a technology like radio frequency identification (RFID).

Another aspect of the present invention in at least one embodiment is the need to avoid sticking of at least portions of incision edges 12 or 13 to the lower surface 15. In order to avoid such sticking vacuum ports 23 may be configured to deliver positive pressure to push the tissue away from the wedge body 10. In yet another embodiment, cooling elements embedded in the wedge body volume which are configured to cool area 22 may be also configured to deliver heat to adjacent tissue upon user command to allow easier separation and relocation of treatment wedge 10 with minimal mechanical tear forces applied on the incision which may cause bleeding or opening of the incision.

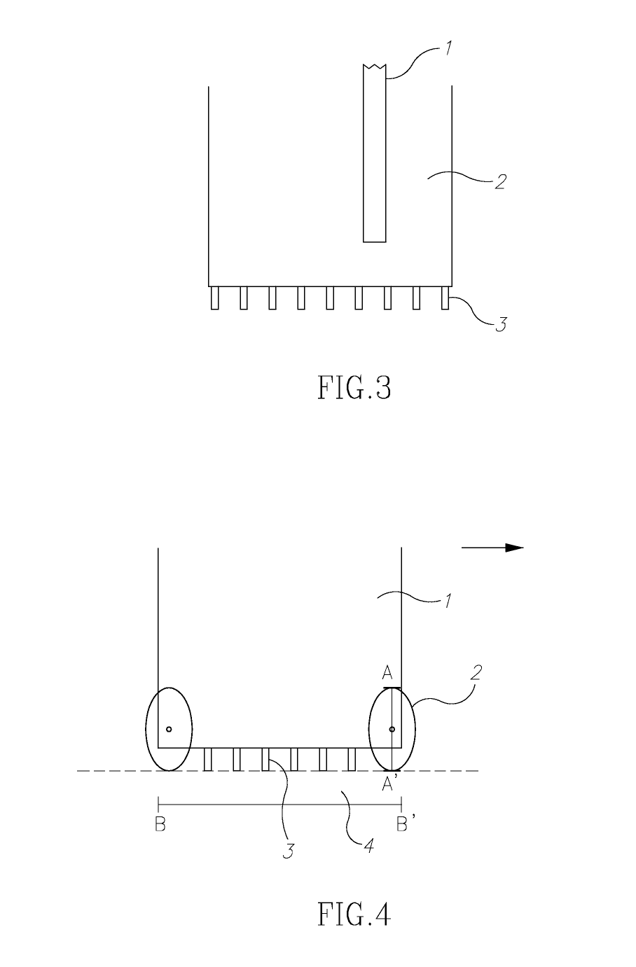

FIG. 3 illustrates another aspect of the present invention in which, at least in one embodiment, a hot body is disclosed. The hot body may comprise a thermal mass element 2 and a heat source 1 coupled to the thermal mass element 2. The thermal mass element 2 has a fractional surface 3. A fractional surface 3, in accordance to one embodiment of the present invention, may mean a relatively flat surface with at least one protrusion which is extended from the flat surface. In yet another embodiment of the present invention, multiple protrusions in multiple shapes, sizes or distribution densities may extend form the thermal mass element surface.

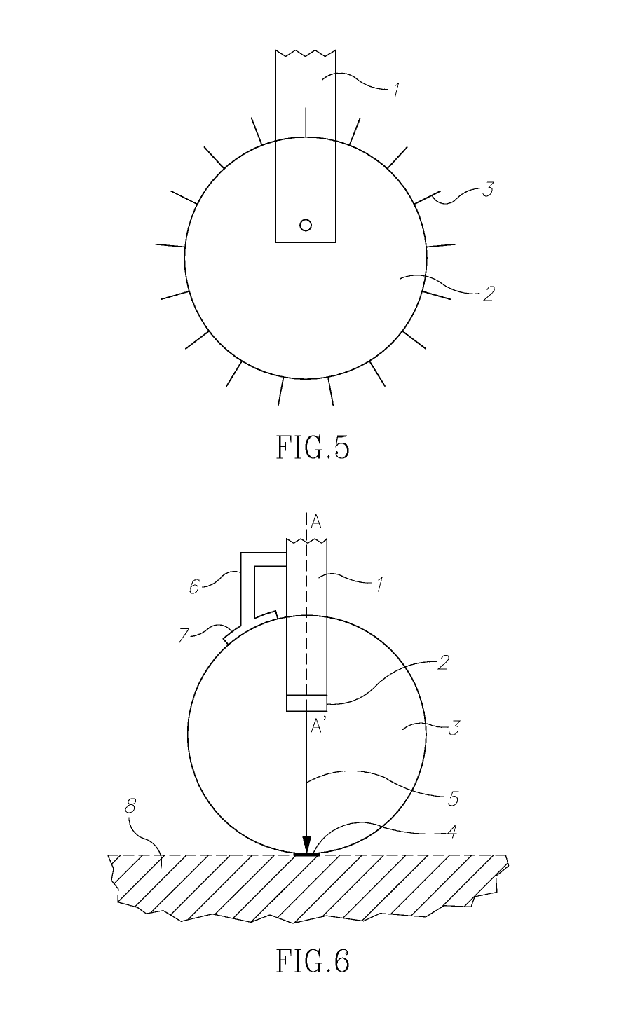

In yet another configuration, the thermal mass element surface 2 is circular, as shown in FIG. 5. The thermal mass element 2 and heat source 1 may be integrated, in accordance to one embodiment of the invention, into a single element. The hot body, according to one aspect of the present invention, is configured to stamp a fractional heat pattern on a target tissue. According to one embodiment, a hot tip system may be a hand-held pen-like and stand-alone system which manually stamps fractional heat patterns on different locations on the target tissue. In yet another embodiment, the hot tip system may be configured to have at least one rotatable element 2 as shown in FIGS. 4 and 5. These rotatable elements, under reasonable friction with the target tissue, may be configured to provide a continuous fractional heat treatment as shown in FIG. 5 or a semi-continues, step and stamp, mode of operation, as further disclosed for example, in FIG. 4, in accordance to one embodiment of the invention. In one embodiment, at least one eccentric ellipsoid wheel 2 is attached to the hot tip system 1. Eccentric wheel 2 is configured to contact target tissue 4. Moving the hot tip by the user along a treatment direction, while maintaining a reasonable degree of friction between the target tissue and the rotatable element, the hot tip propagates along the target tissue. Eccentric ellipsoid has a semi-major-axis AA′. BB′ defines the hot tip width. Along a rotation of the eccentric ellipsoid 2, the fractional surface 3 of the hot tip 1 will be raised from the target tissue 4 and will impinge again on a different location on the target tissue 4, after the ellipsoid concludes a full 360 degree rotation. This relocation of the fractional surface is defined as the step the hot tip makes before it stamps again another portion of the tissue.

In yet another embodiment of the invention, the wheel 3 or shielding element may have other optical elements than the optical aperture, to enhance the treatment and more effectively use the optical energy emitted from the at least one light source 2. Such optical element may include lenses, which focus emitted light to increase energy fluence as it reaches the target tissue.

FIG. 6 illustrates another embodiment of the present invention comprising a shaft 1. According to this embodiment, at least one light source 2 is attached to the shaft 1. The at least one light source 2 can be attached directly or indirectly to the shaft 1. In yet another embodiment of the invention, the at least one light source 2 can be aligned with the main axis of the shaft AA′. In yet another embodiment, the at least one light source may be connected to the shaft along an axis which is off-set from the main axis of the shaft AA′. In one embodiment of the present invention, at least a portion of the shaft 1, including the portion to which the at least one light source 2 is attached, is accommodated within a wheel 3. In yet another embodiment, the entire shaft 1 is located external to the wheel 3 and only the at least one light source 2 is accommodated within the wheel 3. The light source may comprise a laser light.

According to one aspect of the present invention, the at least one light source 2 is configured to be in an optical communication with a target tissue 8. Target tissue 8 may be located approximately below the shaft 1. In one embodiment, the target tissue 8 is located along the main axis AA′ of the shaft 1. In yet another configuration, the target tissue 8 may be located along an off-set axis to the main axis AA′ of the shaft. In one embodiment, wheel 3 is rotatably connected to shaft 1. According to one embodiment, while wheel 3 rotates, shaft 1 and attached at least one light source 2, propagate in a direction to which the wheel rotates. In one embodiment of the present invention, the system is configured to propagate the at least one light source 2 along a target tissue.

According to another aspect of the present invention, in one embodiment, wheel 3 consists of at least one aperture 4 which rotates with wheel 3. In this embodiment, the at least one aperture is configured to cyclically establish an optical communication between the at least one light source 2 and the target tissue 8. In another embodiment of the present invention, the wheel 3 may comprise multiple such apertures which are configured to establish an optical communication between single or multiple light sources and multiple spots on the target tissue 8. According to embodiment, while the user pushes the handpiece along the target tissue 8 while maintain continuous reasonable friction between the target tissue 8 and wheel 3, the at least one light source 2 will propagate along a plane relatively parallel to the target tissue 8 and due the cyclically rotating at least one aperture 4, a trace of fractional laser treatment will be delivered to target tissue 8.

In yet according to another aspect of the present invention a cleaning element 6 may be connected to shaft 1. Cleaning element 6 may have, according to another embodiment, a contacting element 7 which is configured to be in continuous contact with at least a portion of wheel 3. In one embodiment, the contacting element 7 is positioed to wipe cyclically rotating the at least one aperture, in order to clean the aperture, after contact with the target tissue, to allow effective optical communication between the at least one light source and the at least one aperture once this apertures reaches an alignment with such a light source. Cleaning element 6 may comprise multiple contacting elements 7.

The contacting element 7, according to one embodiment, is a disposable element which can be replaced by the user if and when it become ineffective.

In another aspect of the present invention, in order to reduce costs and increase patient safety, the wheel is a replaceable single patient element. In another embodiment, the wheel and the cleaning element are disposable and replaceable elements while the shaft and the at least one light source are reusable elements which are protected and do not contact the patient directly. In yet another configuration of the present invention, the entire system may be reusable and a shielding element is applied to the wheel. In a further embodiment, the shielding element may also have structural or optical characteristic instead of, or in addition to, structural or optical characteristics of the wheel.

In another aspect of the present invention there is provided a treatment method to prevent scar formation. The method of treatment, according to one embodiment of the present invention is to fractionally treat at least a portion of an incision edge prior to closure of the incision. In the prior art, one way to close a surgical incision is by stitching. Where thick tissue is involved, like with abdominal surgeries, there is a need first to stitch the lower layers of the tissue. Only then the physician brings together the outer layers and stitches them in order to close the incision. According to one aspect of the present invention, a fractional treatment of at least a portion of the incision edges may result in decreased scarring and better aesthetic outcome. The fractional treatment may be delivered before or after the first stitching.

In the above description, an embodiment is an example or implementation of the invention. The various appearances of “one embodiment”, “an embodiment” or “some embodiments” or “embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Furthermore, it is to be understood that the invention can be carried out or practiced in various ways and that the invention can be implemented in embodiments other than the ones outlined in the description above.

The invention is not limited to those diagrams or to the corresponding descriptions. For example, flow need not move through each illustrated box or state, or in exactly the same order as illustrated and described.

Meanings of technical and scientific terms used herein are to be commonly understood as by one of ordinary skill in the art to which the invention belongs, unless otherwise defined.

While the invention has been described with respect to a limited number of embodiments, these should not be construed as limitations on the scope of the invention, but rather as exemplifications of some of the preferred embodiments. Other possible variations, modifications, and applications are also within the scope of the invention.

1. A system for treating incisions, for the prevention of scar tissue, the system comprising:
a wedge body;
the wedge body having an upper surface, a lower surface and a volume;
wherein the lower surface is configured to contact at least a portion of an incision edge; and
wherein the wedge body volume is configured to deliver treatment energy through the lower wedge surface to adjacent incision edges.

2. The system according to claim 1, wherein the lower surface of the edge defines a first region and a second region and wherein the first region is configured to contact at least a portion of a first incision edge and the second region is configured to contact at least a portion of a second incision edge.

3. The system according to claim 2, wherein the first incision edge and the second incision edge are at least partially opposite to each other.

4. The system according to claim 1, wherein the wedge body volume comprises at least one energy source.

5. The system according to claim 4, wherein the at least one energy source is configured to deliver treatment energy to at least a portion of an incision edge through the lower surface of the wedge body.

6. The system according to claim 4, wherein the at least one energy source is one of a diode laser, a radio frequency electrode, an ultrasound transducer or a needle electrode.

7. (canceled)

8. (canceled)

9. (canceled)

10. The system according to claim 4, wherein the at least one energy source is an array of multiple energy sources.

11. The system according to claim 10, wherein the array of multiple energy sources are configured to create a fractional treatment to at least a portion of the tissue contacting the lower surface.

12. The system according to claim 1, wherein the lower wedge surface is further configured to pull at least a portion of the incision edge.

13. The system according to claim 12, wherein the lower wedge surface is configured to pull the at least portion of the incision edge through at least one vacuum channel.

14. The system according to claim 11 wherein the wedge body volume further comprises a cooling mechanism which is configured to cool the tissue between the fractional treatment spots.

15. (canceled)

16. (canceled)

17. (canceled)

18. (canceled)

19. (canceled)

20. (canceled)

21. (canceled)

22. (canceled)

23. (canceled)

24. (canceled)

25. An incision treatment device, comprising: a thermal mass element having a fractional surface; a heat source;
wherein the heat source is thermally coupled to the thermal mass element; and wherein with the accumulation of heat within the thermal mass element, the thermal mass element is configured to stamp a fractional heat treating pattern on a target tissue.

26. An device according to claim 25, wherein the device is a stand-alone handheld device.

27. The device according to claim 25, wherein the fractional surface of the thermal mass element further comprises multiple protrusions.

28. The device according to claim 27, wherein the multiple protrusions further comprise micro needles.

29. The device according to claim 25, wherein the device further comprises at least one rotatable element which is configured to contact the target tissue.

30. The device according to claim 29, wherein the rotatable element is an eccentric ellipsoid or a wheel.

31. (canceled)

32. The device according to claim 30, wherein the rotatable element is the thermal mass element.

33. The device according to claim 32, wherein the rotatable thermal mass element further comprises multiple protrusions.
